(19) United States
(12) Patent Application Publication
(10) Pub. No.: US 2024/0009359 A1
(43) Pub. Date: Jan. 11, 2024

(54) DRUG ELUTING ELASTIC BANDS AND LIGATION

(71) Applicant: MEMORIAL SLOAN KETTERING CANCER CENTER, New York, NY (US)

(72) Inventors: Ron Feiner, New York, NY (US); Takeshi Irie, New York, NY (US); Daniel A. Heller, New York, NY (US)

(21) Appl. No.: 18/044,017

(22) PCT Filed: Aug. 30, 2021
(86) PCT No.: PCT/US2021/048203

§ 371 Date: Mar. 3, 2023

Related U.S. Application Data
(60) Provisional application No. 63/074,714, filed on Sep. 4, 2020.
(60) Provisional application No. 63/155,393, filed on Mar. 2, 2021.

(51) Int. Cl. A61L 31/16; A61L 31/04

(52) U.S. Cl. CPC A61L 31/16; A61L 31/049

(57) ABSTRACT

Described herein are medical apparatuses, formulations, manufacturing processes, and medical procedures relating to drug eluting rubber bands that may be used for banding procedures such as hemorrhoid ligation. Examples of therapeutic agents that can be eluted for therapeutic effect include local anesthetics, opioids, calcium channel blockers, antibiotics, sclerosing agents, and steroids.

### STATEMENT OF RELATED APPLICATIONS

This application claims the benefit of and priority as a PCT Application to U.S. Provisional Patent Application No. 63/074,714, filed Sep. 4, 2020, and U.S. Provisional Patent Application No. 63/155,393, filed Mar. 2, 2021, the entire contents of which are incorporated herein by reference.

### TECHNICAL FIELD

The present application relates generally to devices, formulations, manufacturing processes, and medical procedures and treatments relating to drug eluting elastic bands suited to medical banding such as hemorrhoid ligation, and may be implemented as elastic bands comprising composites of elastomers (e.g., natural rubber or other elastic polymers) and therapeutic agents that may be used to elute (e.g., locally release through diffusion) therapeutic agents (e.g., anesthetics, opioids, calcium channel blockers, antibiotics, sclerosing agents, and/or steroids) into tissues and their surroundings.

### BACKGROUND

Medical banding procedures use elastic bands to constrict various physiological structures. Banding may be performed to restrict blood flow to ligated tissues, so that the tissue eventually necroses and breaks away from supporting tissue, or to otherwise change how an organ functions without necrosis. Rubber band ligation is used to treat internal hemorrhoids by applying a small band to the base of the hemorrhoid above the dentate line, cutting of blood supply to the hemorrhoidal mass with the goal of having the withered hemorrhoid fall off a number of days later. Rubber band ligation is amongst the most frequently practiced treatments for symptomatic internal hemorrhoids and is performed routinely and frequently by colorectal surgeons.

While the use of rubber band ligation for internal hemorrhoids is well established, treatment of external hemorrhoids by this procedure is historically avoided due to the high level of pain experienced after banding of external hemorrhoids. This anatomic distinction of internal versus external hemorrhoids arises from the differences in innervation of the internal versus the external anus, delineated by the dentate line. The external aspects of the anus (external to the dentate line) are more extensively innervated by pain sensory nerves than in the internal aspect of the anus (above the dentate line), where internal hemorrhoids arise. Historically, only internal hemorrhoids have been amenable to rubber band ligation due to pain. Systemic analgesics are not well-suited to address the pain, as such analgesics are not sufficiently long-lasting, and can be problematic (if even available as an option for any particular patient) for numerous pharmacological reasons, potentially resulting in undesirable systemic effects.

### SUMMARY

One aspect of the present disclosure is directed to a drug-eluting elastic band. Another aspect of the present disclosure is directed to a process for manufacturing drug-eluting elastic band. Yet another aspect of the present disclosure is directed to medical procedures involving a drug-eluting elastic band.

Various embodiments of the disclosure relate to a drug-eluting elastic medical apparatus comprising a composite of a therapeutic agent (or a plurality of therapeutic agents) and an elastomer (or a plurality of elastomers). The medical apparatus may be manufactured by dissolving the elastomer and the therapeutic agent(s) in an organic solvent (or a plurality of organic solvents), and evaporating the organic solvent(s) while the elastomer reforms into the composite having a shape.

In various embodiments, the organic solvent may be evaporated while the elastomer reforms in a mold corresponding to the shape. The shape may correspond to a rubber band. The rubber band may comprise an outer diameter of 4 to 6 millimeters (mm), an inner diameter of 1 to 2 mm, and/or a height of 1.5 to 2.5 mm. For example, the outer diameter may be about 5 mm, the inner may be about 1.5 mm, and the height may be about 2 mm.

In various embodiments, the shape may correspond to a 3D printer filament for a 3D printer. The medical apparatus may further be manufactured using the 3D printer filament in a 3D printer to print the medical apparatus (e.g., an elastic band).

In various embodiments, the elastomer may be a rubber composite. The elastomer may comprise polystyrene-block-polyisoprene-block-polystyrene (SIS). The elastomer may range from about, for example, 15 percentage by weight (wt %) polystyrene to about 40 wt % polystyrene. The elastomer may be, for example, about 22 wt % polystyrene. The elastomer may consist substantially of a combination of at least two of (i) polyisoprene or derivatives thereof, (ii) polystyrene or derivatives thereof, (iii) polyisoprene or derivatives thereof, (iv) polyurethane or derivatives thereof, and (v) silicone or derivatives thereof.

In various embodiments, the therapeutic agent may comprise, for example, an analgesic, an anti-inflammatory agent, an anti-microbial agent, and/or a sclerotic agent.

In various embodiments, the composite comprises a combination of two or more therapeutic agents. The combination may comprise, for example, an analgesic and an anti-inflammatory agent.

In various embodiments, the shape may be a first shape, and the medical apparatus may be further manufactured by heating the composite to at least a melting temperature of the composite, and dye-casting the composite into a second shape.

In various embodiments, the medical apparatus may comprise a rubber band manufactured to release the therapeutic agent through diffusion from pores in the rubber band.

In various embodiments, the medical apparatus may be configured to be used in a medical procedure that brings the medical apparatus into contact with a tissue of a subject to locally release the therapeutic agent into the tissue and/or its surroundings. The tissue may be an outgrowth. The medical procedure may strangulate and necrose the tissue such that the tissue breaks off. Alternatively or additionally, the medical procedure may release the therapeutic agent for uptake by the tissue or its surroundings to cause a therapeutic effect.

In various embodiments, the shape may correspond to a rubber band. The medical procedure may comprise banding of a hemorrhoid. The hemorrhoid may be an internal hemorrhoid or an external hemorrhoid.

In various embodiments, the medical apparatus may further comprise an elastic band applicator (such as a McGivney hemorrhoid ligator), a vacuum probe, and/or forceps.

Various embodiments of the disclosure relate to a medical procedure comprising securing a drug-eluting elastic medical apparatus to a tissue of a subject to locally release a therapeutic agent (or a plurality of therapeutic agents) into the tissue and/or its surroundings. The medical apparatus may comprise a composite of the therapeutic agent (or the plurality of therapeutic agents) and an elastomer (or a plurality of elastomers). The medical apparatus may be manufactured by dissolving the elastomer(s) and the therapeutic agent(s) in an organic solvent (or a plurality of organic solvents), and evaporating the organic solvent(s) while the elastomer reforms into the composite having a shape.

In various embodiments, the tissue may be an outgrowth. The medical procedure may strangulate and necrose the tissue such that the tissue breaks off. Alternatively or additionally, the medical procedure may release the therapeutic agent for uptake by the tissue or its surroundings to cause a therapeutic effect.

In various embodiments, the shape may correspond to a rubber band. The medical procedure may comprise banding of a hemorrhoid. The hemorrhoid may be an internal hemorrhoid or an external hemorrhoid.

In various embodiments, the organic solvent may be evaporated while the elastomer reforms in a mold corresponding to the shape. The shape may correspond to a rubber band. The rubber band may comprise an outer diameter of 4 to 6 millimeters (mm), an inner diameter of 1 to 2 mm, and/or a height of 1.5 to 2.5 mm. For example, the outer diameter may be about 5 mm, the inner may be about 1.5 mm, and the height may be about 2 mm.

In various embodiments, the shape may correspond to a 3D printer filament for a 3D printer. The medical apparatus may be further manufactured by using the 3D printer filament in the 3D printer to print the medical apparatus.

In various embodiments, the elastomer may be a rubber composite. The elastomer may comprise polystyrene-block-polyisoprene-block-polystyrene (SIS). The elastomer may range from about 15 percentage by weight (wt %) polystyrene to 40 wt % polystyrene. The elastomer may be, for example, about 22 wt % polystyrene. The elastomer may consist substantially of a combination of at least two of (i) polyisoprene or derivatives thereof, (ii) polystyrene or derivatives thereof, (iii) polyisoprene or derivatives thereof, (iv) polyurethane or derivatives thereof, and (v) silicone or derivatives thereof.

In various embodiments, the therapeutic agent may comprise, for example, an analgesic, an anti-inflammatory agent, an anti-microbial agent, and/or a sclerotic agent. The composite may comprise a combination of two or more therapeutic agents, such as a combination that includes an analgesic and an anti-inflammatory agent.

In various embodiments, the shape may be a first shape, and the medical apparatus may be further manufactured by heating the composite to at least a melting temperature of the composite, and dye-casting the composite into a second shape.

In various embodiments, the medical apparatus may comprise a rubber band manufactured to release the therapeutic agent through diffusion from pores in the rubber band.

Various embodiments of the disclosure relate to a method comprising manufacturing a drug-eluting elastic medical apparatus comprising a composite of a therapeutic agent (or a plurality of therapeutic agents) and an elastomer (or a plurality of elastomers) by dissolving the elastomer(s) and the therapeutic agent(s) in an organic solvent (or a plurality of organic solvents), and evaporating the organic solvent(s) while the elastomer reforms into the composite having a shape.

In various embodiments, the organic solvent may be evaporated while the elastomer reforms in a mold corresponding to the shape. The shape may correspond to a rubber band. The rubber band may comprise an outer diameter of 4 to 6 millimeters (mm), an inner diameter of 1 to 2 mm, and/or a height of 1.5 to 2.5 mm. For example, the outer diameter may be about 5 mm, the inner may be about 1.5 mm, and the height may be about 2 mm.

In various embodiments, the shape may correspond to a 3D printer filament. Manufacturing the medical apparatus may further comprise using the 3D printer filament to print the medical apparatus.

In various embodiments, the elastomer may be a rubber composite. The elastomer may comprise polystyrene-block-polyisoprene-block-polystyrene (SIS). The elastomer may range from about 15 percentage by weight (wt %) polystyrene to 40 wt % polystyrene. The elastomer may be, for example, about 22 wt % polystyrene. The elastomer may consist substantially of a combination of at least two of (i) polyisoprene or derivatives thereof, (ii) polystyrene or derivatives thereof, (iii) polyisoprene or derivatives thereof, (iv) polyurethane or derivatives thereof, and (v) silicone or derivatives thereof.

In various embodiments, the therapeutic agent may comprise, for example, an analgesic, an anti-inflammatory agent, an anti-microbial agent, and/or a sclerotic agent.

In various embodiments, the composite comprises a combination of two or more therapeutic agents. The combination may comprise, for example, an analgesic and an anti-inflammatory agent.

In various embodiments, the shape is a first shape, and manufacturing the medical apparatus may further comprise heating the composite to at least a melting temperature of the composite, and dye-casting the composite into a second shape.

In various embodiments, the medical apparatus comprises a rubber band that is manufactured to release the therapeutic agent through diffusion from pores in the rubber band.

In various embodiments, the method further comprises using the medical apparatus in a medical procedure that brings the medical apparatus into contact with a tissue of a subject to locally release the therapeutic agent into the tissue and/or its surroundings. The tissue may be an outgrowth. The medical procedure may strangulate and necrose the tissue such that the tissue breaks off. Alternatively or additionally, the medical procedure may release the therapeutic agent for uptake by the tissue or its surroundings to cause a therapeutic effect.

In various embodiments, the shape may correspond to a rubber band. The medical procedure may comprise banding of a hemorrhoid using the rubber band. The hemorrhoid may be an internal hemorrhoid or external hemorrhoid.

These and other features of various embodiments can be understood from a review of the following detailed description in conjunction with the accompanying drawings.

It is to be understood that both the foregoing general description and the following detailed description and accompanying drawings are exemplary and explanatory and are not restrictive of the present invention, as claimed.

### BRIEF DESCRIPTION OF THE FIGURES

The foregoing and other objects, aspects, features, and advantages of the disclosure will become more apparent and better understood by referring to the following description taken in conjunction with the accompanying drawings, in which:

FIG. 1 provides an example process of manufacturing and using a drug-eluting elastic band in accordance with various potential embodiments.

FIGS. 2A-2B depicts an example drug-eluting elastic band and dimensions thereof in accordance with various potential embodiments.

FIGS. 3A-3E depict surface morphologies analyzed by atomic force microscopy (AFM) of pristine rubber (FIG. 3A) and various composites thereof (2%, 5%, 10%, and 20% drug content W/W % corresponding to FIGS. 3B, 3C, 3D, and 3E, respectively) in accordance with various potential embodiments. FIGS. 3F and 3G depict roughness measurements based on the AFM images of FIGS. 3A-3E.

FIGS. 4A and 4B depict Fourier-transform infrared spectroscopy (FTIR) spectra of pristine rubber (“PolySIS”), pristine lidocaine (“Lidocaine”), and lidocaine loaded within the rubber matrix (“Mix”), with FIG. 4B showing an enlarged version of a region of FIG. 4A, in accordance with various potential embodiments. FIG. 4C depicts FTIR spectra of pristine rubber (“PolySIS”), pristine budesonide (“Budesonide”), and budesonide loaded within the rubber matrix (“Mix”), in accordance with various potential embodiments. No new peaks are discernable in the mixtures, indicating no new chemical interactions. FIG. 4D depicts additional FTIR spectra of pristine rubber, pristine lidocaine, and lidocaine loaded within the rubber matrix, in accordance with various potential embodiments, also showing that no new peaks are discernable in the mixture, indicating no new chemical interactions.

FIG. 5A depicts an example medical apparatus comprising a lidocaine-eluting elastic band loaded onto a medical device to be used as an elastic band applicator (here, a standard McGivney hemorrhoid ligator) which can be used in various banding procedures (such as external hemorrhoid ligation) in accordance with various potential embodiments. FIG. 5B shows an example elastic band stretched over a standard pen.

FIGS. 6A-6C depict an example medical procedure in which a tissue is ligated with a drug-eluting elastic band in accordance with various potential embodiments.

FIGS. 7A-7G depict analyses of mechanical properties of various elastomers in comparison with elastomer composites comprising example therapeutic agents in accordance with various potential embodiments. FIG. 7A shows maximum tensile load compared between pristine polySIS (polystyrene-block-polyisoprene-block-polystyrene (SIS)) and the drug-loaded composites of elastomer and therapeutic agent. FIG. 7B shows percentage strain at break compared between pristine polySIS and the drug-loaded composites. FIGS. 7C and 7D show Young's modulus compared between pristine polySIS and the drug-loaded composites. FIG. 7E shows stress vs strain under cyclic stretching compared between pristine polySIS and the drug-loaded composites. FIG. 7F shows maximum tensile strength compared between the commercial rubber band and polySIS. FIG. 7G shows swelling ratio compared between the commercial rubber band and polySIS.

FIG. 8 depicts cell viability in the presence of an example drug-eluting elastic band in accordance with various potential embodiments, as compared with a control sample and a commercially available rubber.

FIGS. 9A-9L depict release of various therapeutic agents from drug-eluting elastic bands in accordance with various potential embodiments.

FIG. 10 depicts that released drugs remain active after elution from elastomer into media in the presence of MCF-7 cells, indicating that released drug was effective in reducing cell viability when compared to untreated cells, in accordance with various potential embodiments. This figure demonstrates cytotoxicity of salicylic acid on cells when applied directly to cells, versus when released from rubber.

FIGS. 11A-11C show that lidocaine released from rubber bands are active and inhibit spontaneous calcium waves of reporter cells in accordance with various potential embodiments. FIG. 11A depicts snapshots of calcium waves before and after exposure to lidocaine, FIG. 11B provides time series data of the reporter cell line, and FIG. 11C provides amplitude quantification.

FIG. 12 depicts activity of lidocaine released from zebrafish against zebrafish hearts in accordance with various potential embodiments.

FIGS. 13A-13G depict application of drug-loaded rubber bands on ex vivo porcine rectum model, in accordance with various potential embodiments. FIG. 13A depicts rubber bands immediately after application onto the inner intestine. FIGS. 13B-13E depict rubber bands on the intestine after 24 hours. FIG. 13F shows rubber bands after removal from the intestine. FIG. 13G depicts quantification of lidocaine extracted from the banded tissue in FIGS. 13A-13E. More specifically, FIG. 13G shows the amount of lidocaine extracted from the tissue following rubber band removal after 24 hours. The banded tissues were dissected, homogenized and the lidocaine extracted and quantified using HPLC. The figure shows that tissues banded with drug loaded rubber bands contained a significant amount of lidocaine within them while those with pristine or commercially available (both drug free) rubber bands did not contain lidocaine within them.

### DETAILED DESCRIPTION

Following below are more detailed descriptions of various concepts related to, and embodiments of, inventive systems, devices, apparatuses, products, and methods for intramedullary nails and use thereof. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Hemorrhoids are pockets of swollen blood vessels inside the anus. While they can be uncomfortable, they are relatively common in adults and become more frequent with age. They can also be associated with pregnancy, obesity, chronic constipation, lifestyles which increase abdominal straining such as those requiring heavy lifting, consumption of low fiber diets, lifestyles in which rectal tissue can be prone to injury including sexual lifestyles, sedentary lifestyles, and other conditions or states in which gastrointestinal transit is impaired such as irritable bowel syndrome and Crohn's disease. Cancer patients with chronic cancer pain managed by opioids may also experience constipation related to the systemic opioids and suffer hemorrhoids. In many cases, outpatient treatment of hemorrhoids with topical medications is sufficient.

For hemorrhoids unresponsive to outpatient medical treatments, hemorrhoid banding, also called hemorrhoid rubber band ligation, is a relatively safe treatment method. It is a minimally invasive procedure that involves tying the base of the hemorrhoid with a rubber band to stop blood flow to the hemorrhoid. In this method, the hemorrhoid is held in place using a vacuum probe or by forceps, and a special ligator is placed over it. The ligator deploys a rubber band onto the base of the hemorrhoid. The strangulation of the hemorrhoid by the rubber band will ultimately cause the hemorrhoid to necrose and fall off with the rubber band, typically in 10 to 14 days. Pain is the most common complication following the procedure and is usually present for 3 to 10 days following the procedure, although this may vary from very mild pain to severe pain. Local anesthetics can be injected during the procedure; however, their analgesic effects typically last on the order of hours, and the only form of analgesics allowed are over the counter drugs such as acetaminophen or ibuprofen. However, non-steroidal anti-inflammatory drugs (NSAIDs) such as aspirin and ibuprofen may be associated with bleeding. More potent analgesics such as opioids are undesirable for management of this pain in this context, since opioids provoke constipation and thus can exacerbate the condition due to increased straining, and thereby reduce the risk of procedural success.

Rubber band ligation is done routinely and is considered a mainstay and very useful treatment. It is quick and simple and has a high success rate. However, most patients will need more than one banding, and severe pain may deter them from seeking additional treatment which can lead to long term exacerbation. Continuous delivery of local anesthetic through embodiments of the disclosed invention, when applied to various tissues such as external hemorrhoid lesions, may allow unprecedented pain control, allowing patients to tolerate rubber band ligation of external hemorrhoids or other structures. Various embodiments also enable local delivery of other drugs (in addition to analgesics), alone or in combination, to achieve different desired therapeutic effects in various different medical procedures.

Various potential embodiments comprise a co-formulation of therapeutic agents (used interchangeably with “drugs”) and elastomers (used interchangeably with “rubber”) for drug elution to an intended site of action. As used herein, an elastomer refers to natural and/or synthetic polymers having elastic properties. In potential embodiments, drug may be eluted locally at the base of a tissue and/or its surroundings to alleviate pain or impart other therapeutic effects. In example embodiments, a rubber band may be positioned at a base of a hemorrhoid in order to alleviate pain associated with hemorrhoid band ligation, and potentially also to improve therapeutic outcomes for the success of the procedure.

Various potential embodiments deliver drug while also performing the mechanical functions of the rubber band during a medical procedure (such as hemorrhoid ligation). The invention facilitates localized drug delivery, which can afford significant benefits over systemic drug delivery strategies for symptom control. Some benefits of various embodiments include minimization of undesirable side effects associated with systemic dosing of drugs intended for pain control, while achieving and maintaining clinically relevant drug concentrations sufficient for therapeutic effect. For example, elution of local anesthetic can be achieved without concern for local anesthetic systemic toxicity, a dreaded complication of inadvertent intravenous delivery of local anesthetic doses intended for deposition into tissues. Delivery of therapeutic agents to the target site in pregnant patients has the benefit of minimizing systemic drug levels which otherwise might impact fetal growth. As another example, avoiding symptom control via systemic drugs can be particularly helpful for the care of elderly patients, mitigating problems related to polypharmacy and drug-drug interactions that are common with elderly patients. Minimization of systemic drug levels in the care of cancer patients with hemorrhoids is also preferable, to minimize the chance of adverse interaction of drugs intended for hemorrhoid control with anti-cancer therapies (e.g., systemic steroids may impair the efficacy of the immune check point inhibitors). In some implementations, drug cargos with known systemic toxicity or adverse effects may be delivered locally without provoking off-target effects, such as in the delivery of pro-sclerotic agents to the site, thereby achieving local therapeutic effect without triggering systemic toxicities.

Various potential embodiments of a drug-eluting elastic band may be composed of a natural rubber composite, such as a polystyrene-block-polyisoprene-block-polystyrene (“SIS”). In potential embodiments, such a composite may comprise, for example, 22 percentage by weight (wt %) polystyrene. In other implementations, other forms of rubber elastomers may be employed, such as composites with different ratios of polyisoprene and polystyrene, polyisoprene, polyurethane and silicone, and their derivatives.

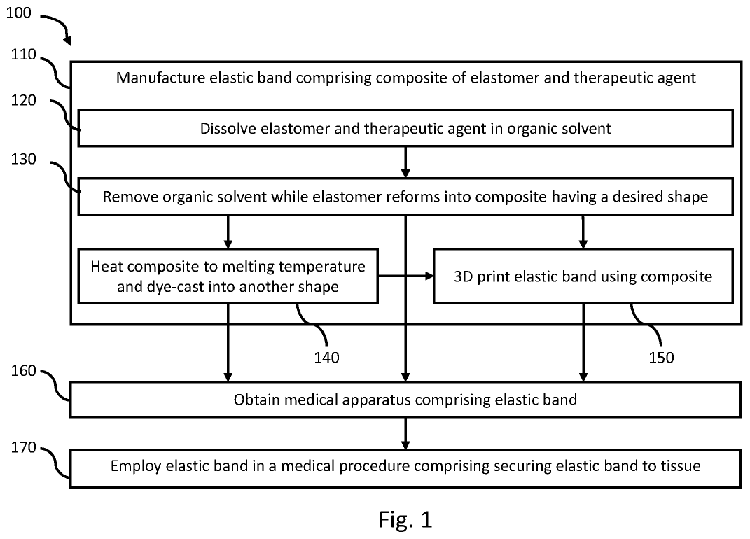

Referring to FIG. 1, a process 100 may comprise manufacturing an elastic band comprising a composite of an elastomer and one or more therapeutic agents (110). In certain embodiments, the elastic band may be pre-manufactured, and the elastic band may be obtained at step 150 (i.e., process 100 may begin at step 150). In some implementations, the drugs may be loaded directly into a rubber matrix of the device by dissolving rubber and the drugs together in one or more organic solvents such as chloroform (120). In various embodiments, a 50% to 90% concentration of the polymer may be mixed with chloroform depending on the mixing instrument used for processing. A combination of drugs may be employed if multiple drugs are to be released from an elastic band. The combination of therapeutic agents to be employed in the rubber-drug composite may be mixed with elastomer in the organic solvent. In certain preferred embodiments, the total concentration of all combinations of drugs may be kept at about 20% W/W or lower so as to maintain desirable mechanical properties of the elastic band. The organic solvent(s) may then be removed while the elastomer reforms into the composite of rubber and drug that has a desired shape (130). For example, the solvent may be evaporated while polymer reforms in a mold. For example, drying of the organic solvent may be performed over a 48-hour period. Temperature may be controlled to facilitate the process, such as heating that aids the evaporation without otherwise negatively impacting the integrity of the components or final product. In various potential embodiments, drug concentrations may range from, for example, 2% weight-by-weight (W/W) to 20% W/W.

In various potential embodiments, in order to reduce air bubbles that interfere with the integrity of the final device, a second dye casting process may be employed whereby, first, the drug and rubber are made into a solution in the organic solvent, and after casting and evaporation in a certain form, the newly formed composite rubber will be heated above its melting temperature and dye cast into the desired shape (140).

Advantageously, through such a manufacturing process (whereby the one or more drugs are mixed with the rubber before casting), any type of shape can be formed. In various examples, the composite may be formed as a filament that may be used in a 3D printer to 3D print a drug-eluting elastomer having a desired shape (150). That is, such an approach could be used for 3D printing of customized devices with controlled drug release properties. In other examples, the composite may be formed to have any other suitable shape or configuration desired for various implementations.

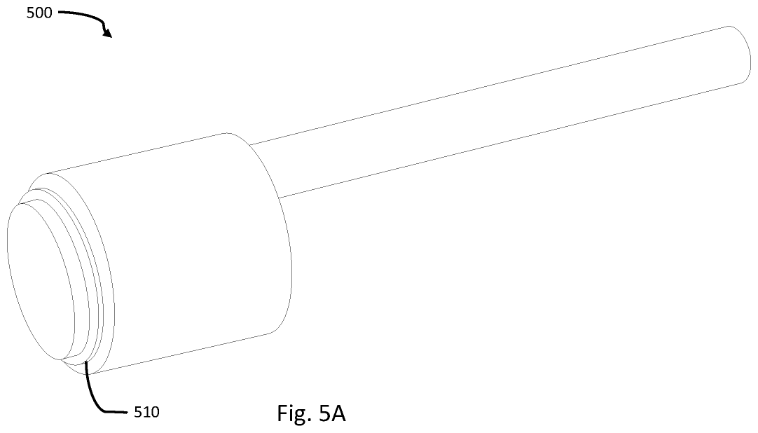

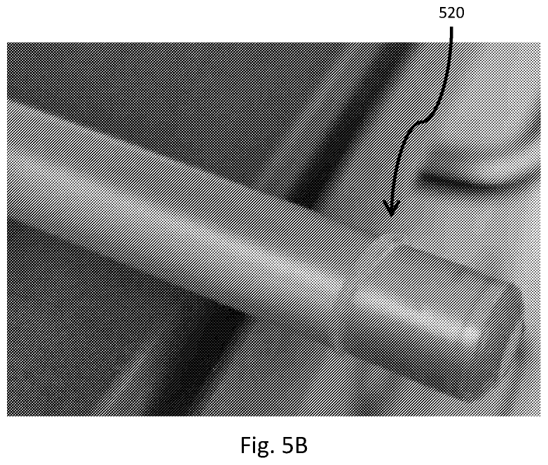

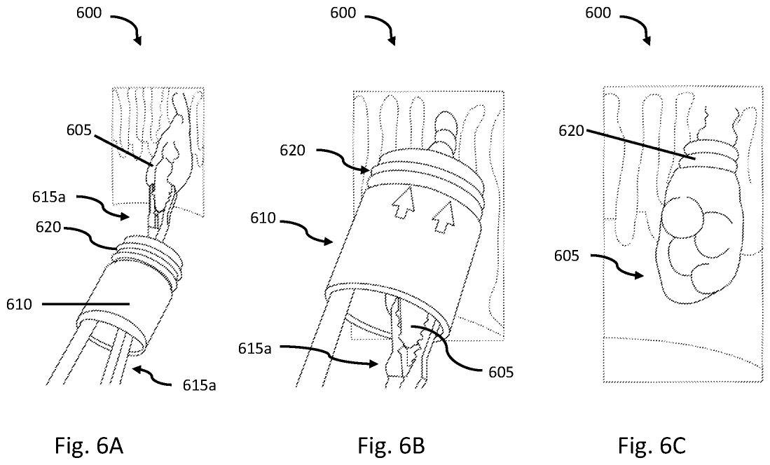

Once the elastic band is manufactured, or otherwise acquired (160), the elastic band may be employed in a medical procedure (170). The medical apparatus may also comprise, for example, one or more surgical instruments that may be used to secure the elastic band to, or otherwise place the elastic band at, a targeted tissue. Example surgical instruments include a McGivney hemorrhoid ligator (see ligator 500, with elastic band 510 loaded thereon, in FIG. 5A), forceps, vacuum probe, etc. With reference to FIGS. 6A-6B, an example surgical procedure 605 involves banding of a tissue 605 (which may be an outgrowth such as an internal or external hemorrhoid) using ligator 610 at which an elastic band 620 is placed. A forceps head 615a extending from a forceps body 615b may be inserted through ligator 610 to grasp the tissue 605 and guide the tissue 605 into the ligator 610 such that elastic band 620 is situated where the tissue 605 is to be strangulated by the elastic band 620. With elastic band 620 in place (FIG. 6C), the instruments may be removed, and the elastic band 620 left in place to necrose the tissue 605 or otherwise cause a therapeutic effect.

Other organic solvents may be employed to form the rubber-drug composite, and temperature modulation during drug mixing and solvent evaporation may be used. In other implementations, drug releasing pellets may be held in place in grooves or pockets in the rubber band. In various embodiments, drug-releasing pellets may be formed from a different polymer loaded with the drug. Example polymers include polylactic co-glycolic acid, polylactic acid, and/or polycaprolactone. Pellets may be, for example, mixed within the rubber or other elastomer during manufacturing. In various preferred implementations, drug release is through diffusion from the pores of the rubber, or through dissolution of the drug releasing pellets. In various embodiments, drug-releasing materials may be biodegradable and may release their content medication through degradation. There may thus be no need for a second coating layer or biodegradable vector.

In various embodiments, other organic solvents that can be employed may include diethyl ether, dichloromethane, acetonitrile and trifluoroethanol, dimethylformamide, and/or toluene. Certain organic solvents may be more suitable to certain elastomers, such as dimethylformamide for polyurethane and toluene for silicone.

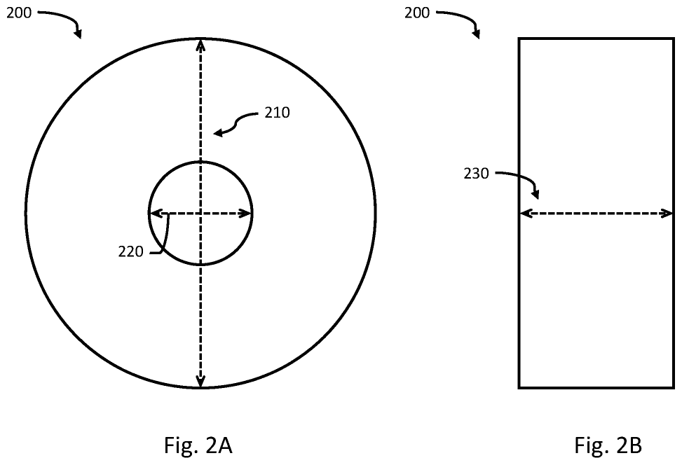

Referring to FIGS. 2A and 2B, example embodiments of the drug-eluting elastic band 200 may have dimensions such as an outer diameter (210) of about 5 millimeters (mm), an inner diameter (220) of about 1.5 mm, and a height (230) of about 2 mm. In other embodiments, the outer diameter may range from, for example, 3 mm to 10 mm, the inner diameter may range from, for example, 0.25 mm to 5 mm, and the height 230 may range from, for example, 0.5 mm to 8 mm. In various embodiments, any toroidal shape with suitable dimensions may be employed, such as for various rubber band ligation (RBL) or other applications. In other embodiments, other shapes and configurations with suitable dimensions may be employed.

An example implementation comprises a rubber band loaded with the anesthetic lidocaine or with the anti-inflammatory steroid budesonide or a combination of the two via the same or similar manufacturing process. Other implementations may contain the non-steroidal anti-inflammatory agent piroxicam or the local anesthetic bupivacaine. Other anesthetics, anti-inflammatory steroids, non-steroidal anti-inflammatory drugs, antibiotics, sclerotic agents, or analgesics may also be loaded into the device for release. Calcium channel blockers such as diltiazem have been shown to have some analgesic effect for hemorrhoidal pain, but such drugs can produce unintended hypotension or cardiac nodal blocking activity, which could be avoided by local diltiazem delivery in some implementations. Potent opioids such as remifentanil, which can be rapidly metabolized by plasma esterases, may also be delivered in some implementations, to achieve analgesic effects at peripheral opioid receptors in the neighborhood, without triggering systemic opioid effects such as sedation, respiratory depression, confusion, and nausea, among others. In certain preferred implementations, all the materials used for the fabrication of the device are FDA approved and the loaded drug quantities are far below toxic systemic levels and may be delivered topically onto a small surface area. In other implementations, novel drugs and elastic polymers not yet FDA approved may also be employed in the manner described.

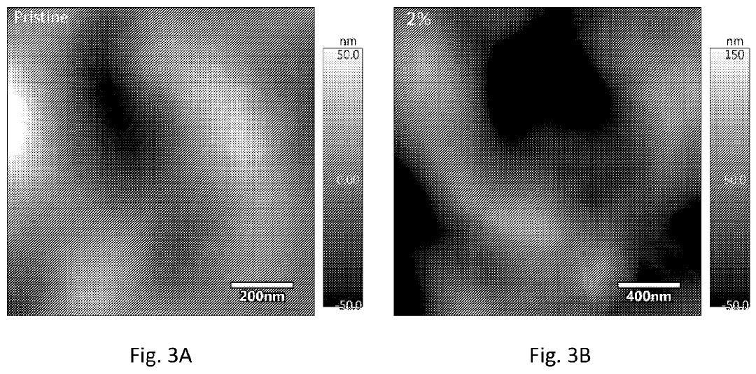

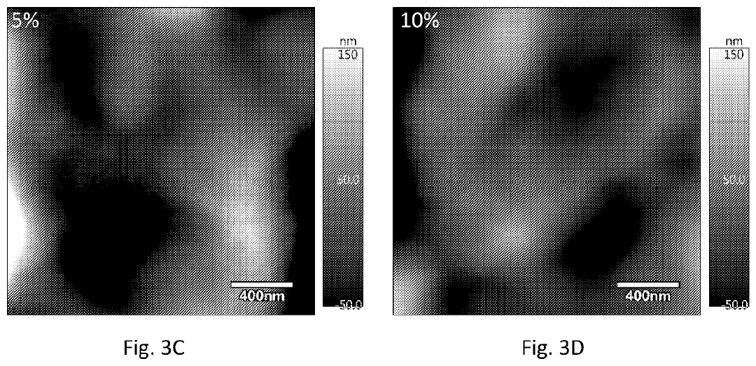

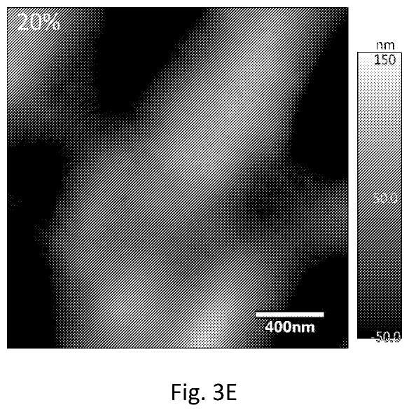

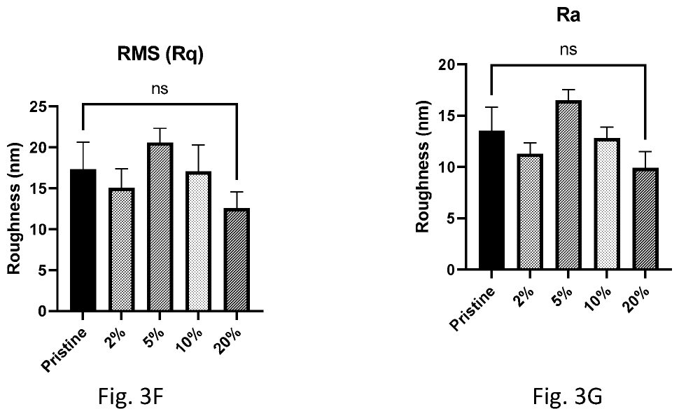

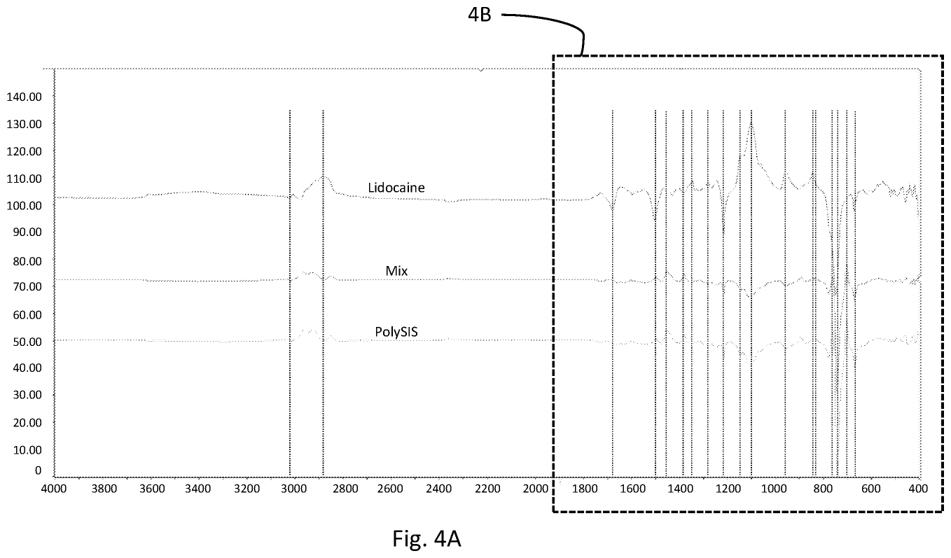

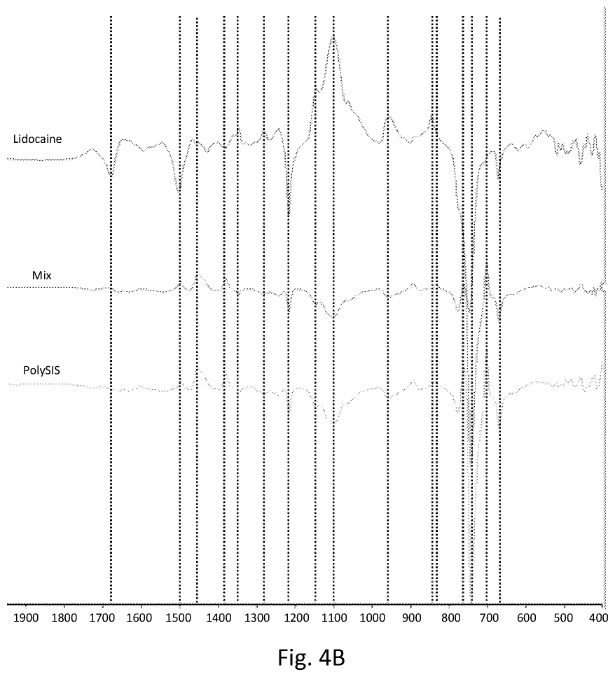

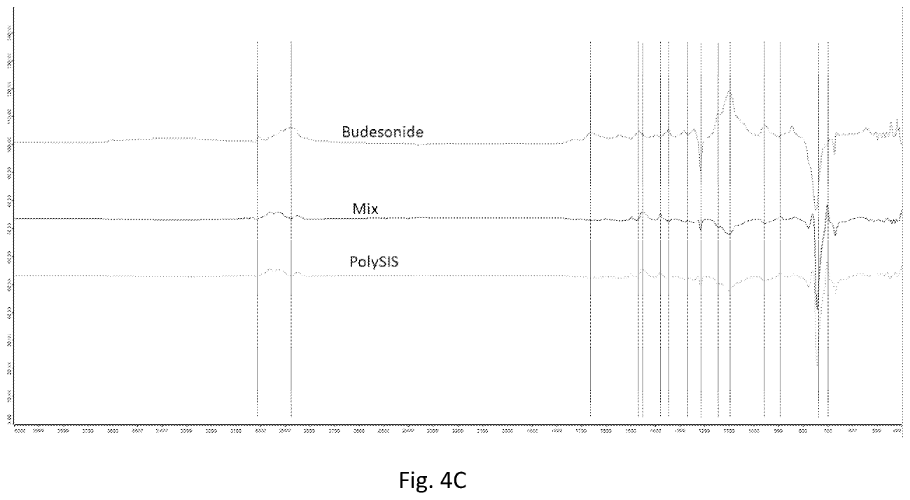

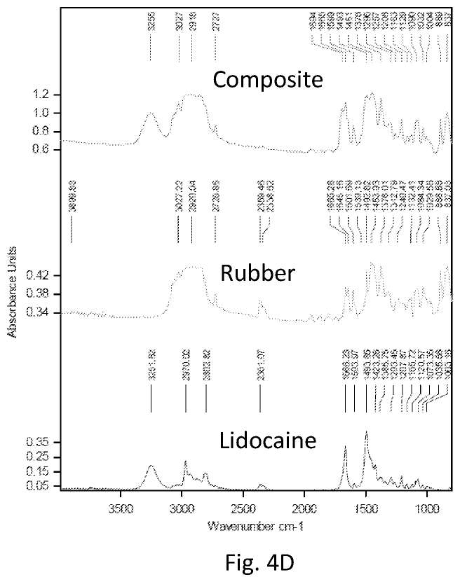

Referring to FIGS. 3A-3E, atomic force microscopy (AFM) shows no clear difference in surface area between the pristine rubber and the surface of the lidocaine loaded rubber ranging from 2% to 20% W/W (FIG. 3). That, forming a composite of elastomer with different concentrations of therapeutic drug ranging from 2% W/W (FIG. 3B) to 20% W/W (FIG. 3E) does not significantly change the surface area as compared with rubber with no drug (FIG. 3A). Surface roughness analyses also showed no significant difference between the pristine rubber and the drug loaded rubber with the highest concentration of loaded drug (FIGS. 3F and 3G). Referring to FIGS. 4A-4D, Fourier-transform infrared spectroscopy (FTIR) clearly shows that the absorption spectrum for the loaded drug (in this case lidocaine or budesonide) is not altered following fabrication, meaning there are no new chemical bonds between the drug and the polymer. This ensures the drug is loaded safely into the polymer matrix and stays functionally intact while in the polymer and when released.

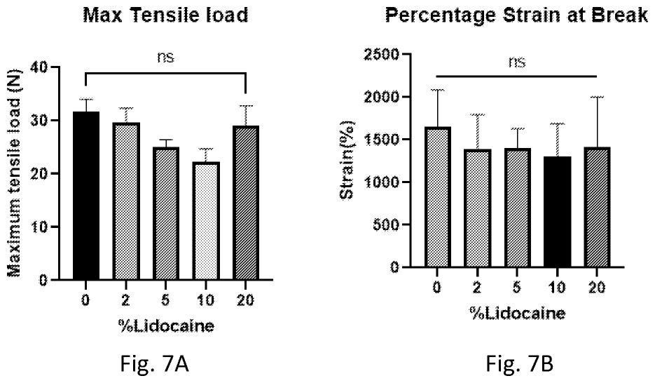

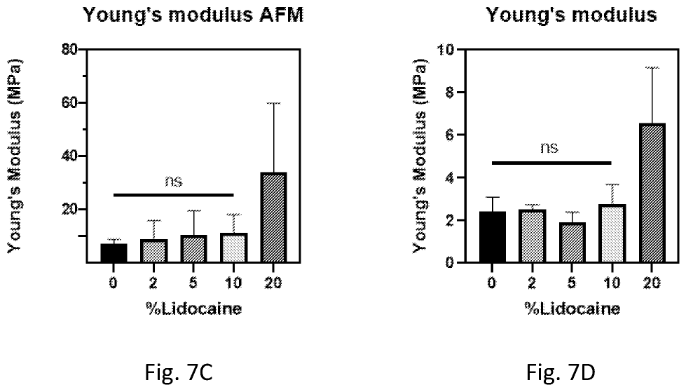

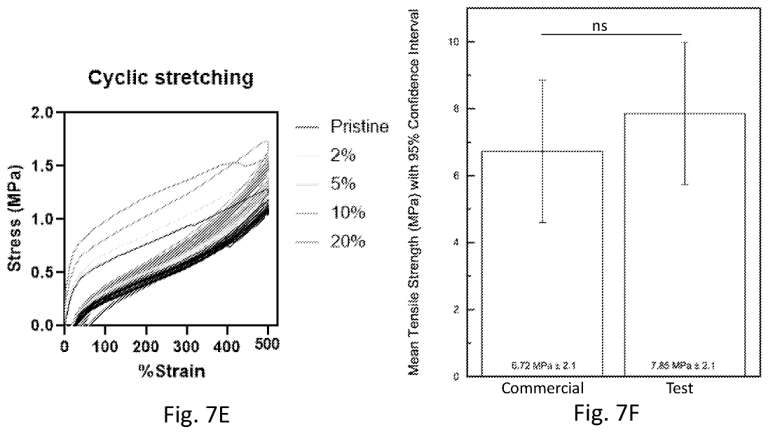

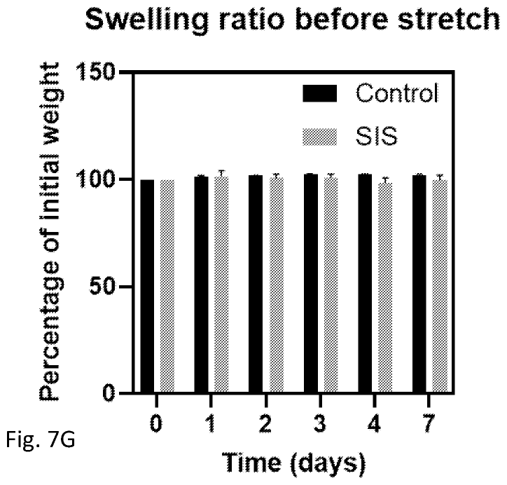

Referring to FIGS. 7A-7G, to show that various potential embodiments of the fabricated lidocaine-loaded rubber band meets the mechanical requirements for the procedure, the rubber band was loaded on a standard McGivney hemorrhoidal ligator without any damage and without rupturing. When rubber band was applied onto a pen as an example (see FIG. 5B), the rubber band (520) returned to its original form following removal. Quantitative mechanical characterization was performed to show that the loaded drug does not interfere with the mechanical function of the device and that it is up to par with a commercially-available conventional rubber band (which does not include eluting drugs). In these tests, it was shown that the maximal tensile load does not change in the rubber when raising the drug concentration to 20% (see FIG. 7A). Additionally, the percentage strain at break, meaning the maximal possible extension the rubber bands can undergo, did not change (see FIG. 7B). The Young's modulus was measured in both atomic force microscopy (see FIG. 7C) and extension through the Instron extension method (see FIG. 7D) and shown to remain the same to at least 10% drug loading. When cyclic stretching was applied onto the rubber for up to 500% (which is well beyond the required extension for various procedures requiring less than 300%), all samples with different drug-loading compositions behaved similarly, with a higher stress built up in the first cycle and then a similar amount of stress over 10 additional cycles (see FIG. 7E). Certain procedures may only require one stretch cycle (stretching during loading and application), though the elastic band is suitable for procedures that may require more than one stretch cycle. When the tensile strength of the rubber used in various potential embodiments was compared to a commercially-available sample, no substantial difference was observed (see FIG. 7F). In addition, potential embodiments of the rubber did not show any significant swelling under rectal physiological conditions (37 degrees Celsius and pH of 7.9) over 7 days (see FIG. 7G) which indicates little to no degradation or change in mechanical integrity over time.

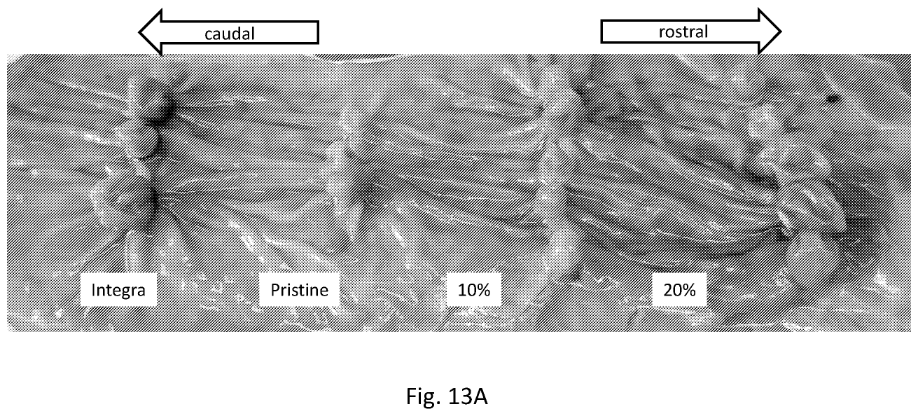

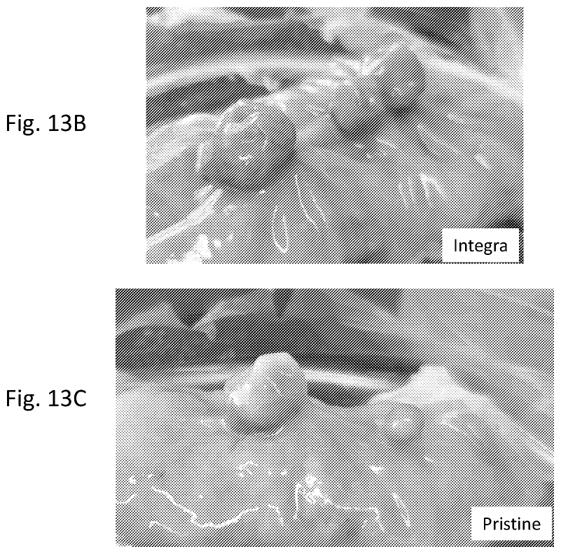

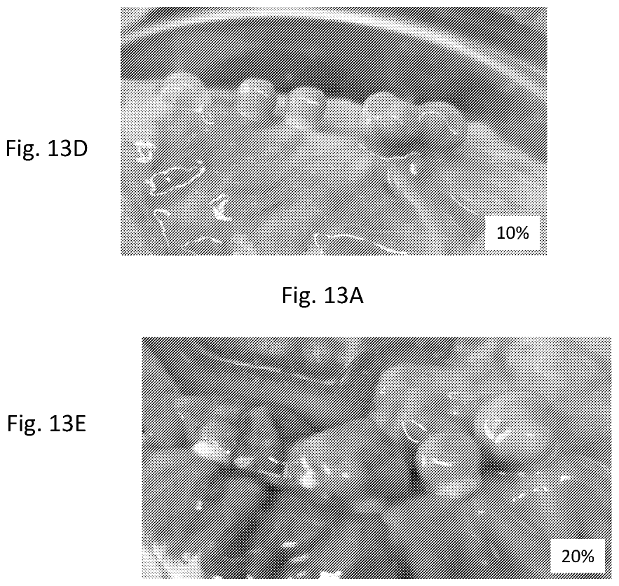

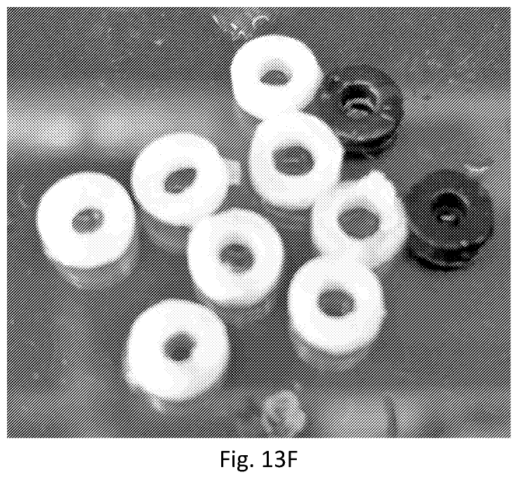

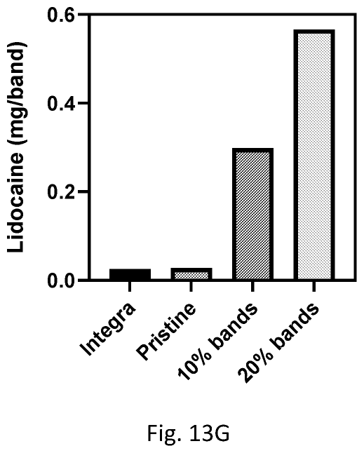

Referring to FIGS. 13A-13F, in accordance with various potential embodiments, application of rubber bands to an ex vivo fresh porcine rectum confirmed that the drug-eluting rubber bands can remain constricted on relevant tissues for 24 hours, further corroborating contractility and mechanical integrity of the drug eluting rubber band. The rubber bands in shown are a commercially-available rubber band (“Integra”), pristine rubber (with no therapeutic agent), along with 10% and 20% loading with lidocaine. Each type of rubber band was shown to be stable on the rectal tissue and showed no sign of buckling, cracking, or tissue escape after 24 hours. The experiment was not continued for more than 24 hours because the sample was fresh and not fixed. After 24 hours, the rubber bands were removed from the tissues, and their gross morphology assessed. Drug eluting rubber bands appeared unchanged at 24 hours, contracting back to their original forms (FIG. 13F). FIG. 13G shows the amount of lidocaine extracted from the tissue following rubber band removal after 24 hours. The banded tissues were dissected, homogenized and the lidocaine extracted and quantified using HPLC. The figure shows that tissues banded with drug loaded rubber bands contained a significant amount of lidocaine within them while those with pristine or commercially available (both drug free) rubber bands did not contain lidocaine within them.

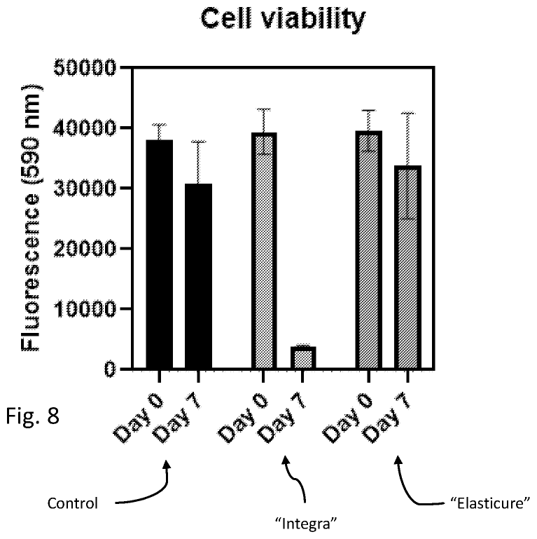

To demonstrate the safety of the rubber used in various potential embodiments of the disclosed device, MCF-7 cells were grown over 7 days in the presence of embodiments of the disclosed rubber band (“Elasticure”), the “Integra” rubber band, and without any sample (control) (FIG. 8). Cell viability in the presence of the “Elasticure” device did not change as compared to the control sample while that of the cells grown in the presence of the commercially-available “Integra” rubber dropped to almost zero after 7 days. This experiment was repeated multiple times in different conformations and all tests showed the same results.

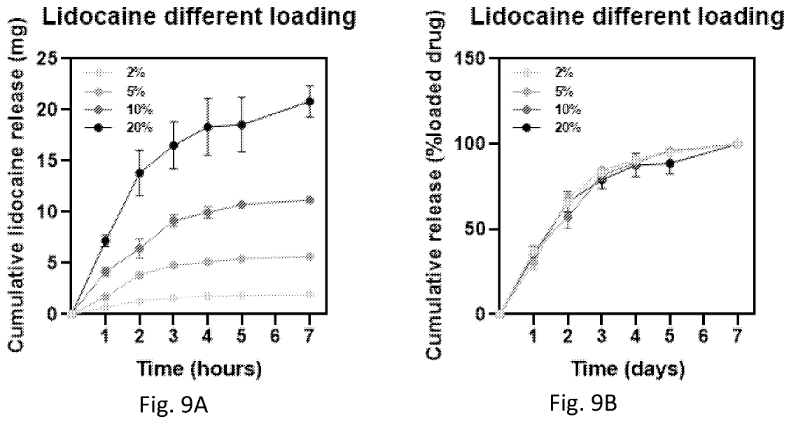

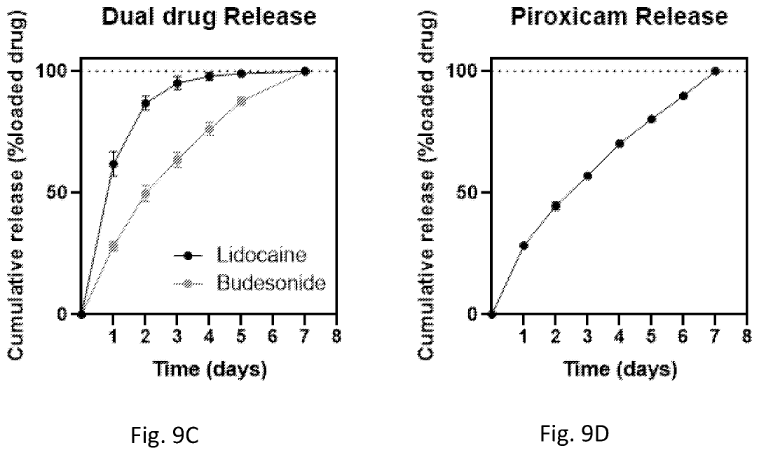

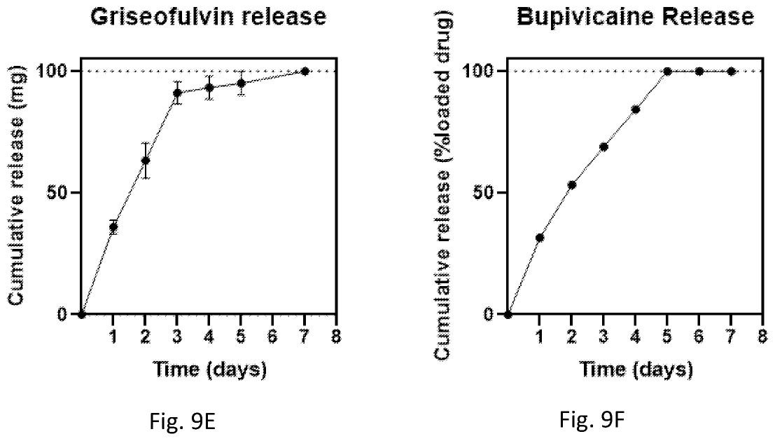

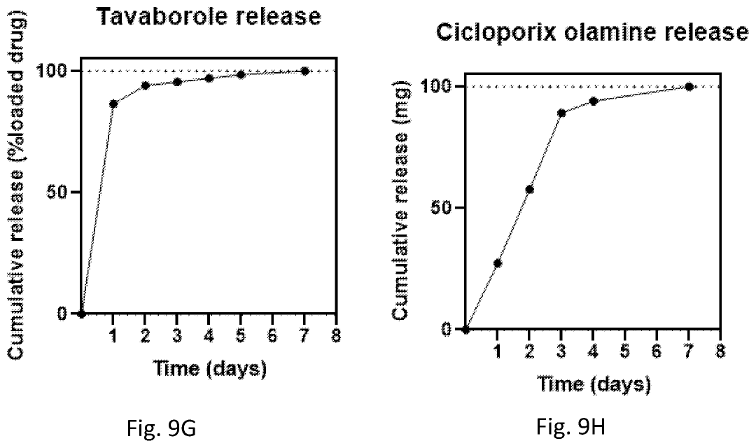

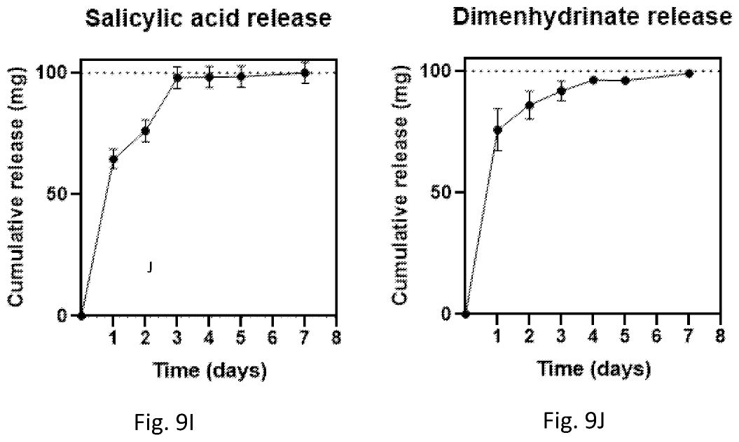

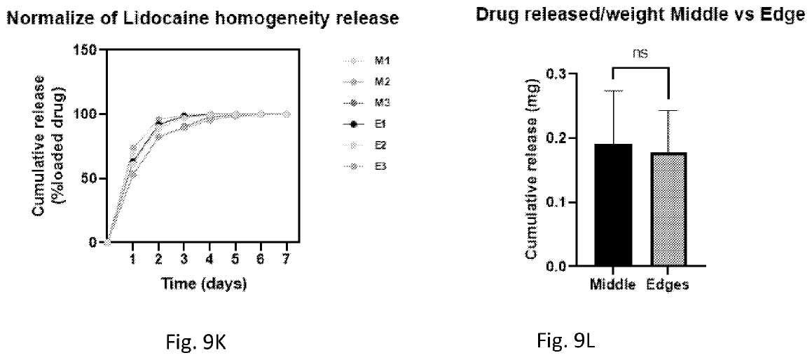

To demonstrate the drug releasing capabilities of various potential embodiments of the disclosed elastic band, rubber bands were placed in physiological medium with a pH of 7.9 and 37 degrees Celsius (similar to that of the rectum) and released drug was measured over time. Lidocaine was loaded at different quantities in the rubber and release was measured over several days and peaked around day 5 (see FIG. 9A). While the amount of lidocaine released varied between the samples according to loading quantity, the release profile remained the same (see FIG. 9B). The initial burst release is both expected and useful as the most significant amount of pain is expected in the days immediately following the procedure. Dual drug release of lidocaine and budesonide was also achieved (see FIG. 9C). Additional drugs released include piroxicam, griseofulvin, bupivacaine, tavabarole, ciclopirox olamine, salicylic acid and dimenhydrinate (see FIGS. 9-9J). To see whether the lidocaine loaded within the rubber is dispersed evenly, a larger lidocaine loaded rubber slab was prepared and cut into pieces from either the middle or the edges of the slab. The amount of drug released was measured (see FIG. 9K) and normalized to the amount of rubber (see FIG. 9L). The results showed that there is no difference in the drug release pattern or in the relative amount of drug released which means drug loading and release is homogenous through the rubber.

Delayed delivery formulations can also be implemented using biodegradable polymers such as PLGA (poly(lactic-co-glycolic acid)), Polylactic acid (PLA) and/or Polycaprolactone (PCL), for example, improve the drug release profile of the device to extend its use, increase or decrease the amount of loaded drug, and vary the drugs and their combination.

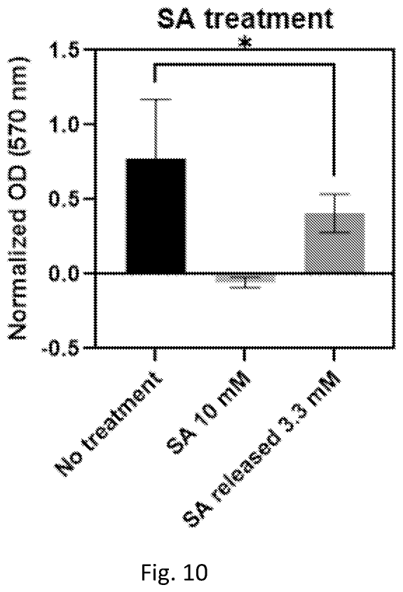

In order to verify that the released drugs are still active after elution from the rubber, salicylic acid was loaded and released into the media in the presence of MCF-7 cells. The released drug was effective in reducing cell viability when compared to untreated cells (see FIG. 10).

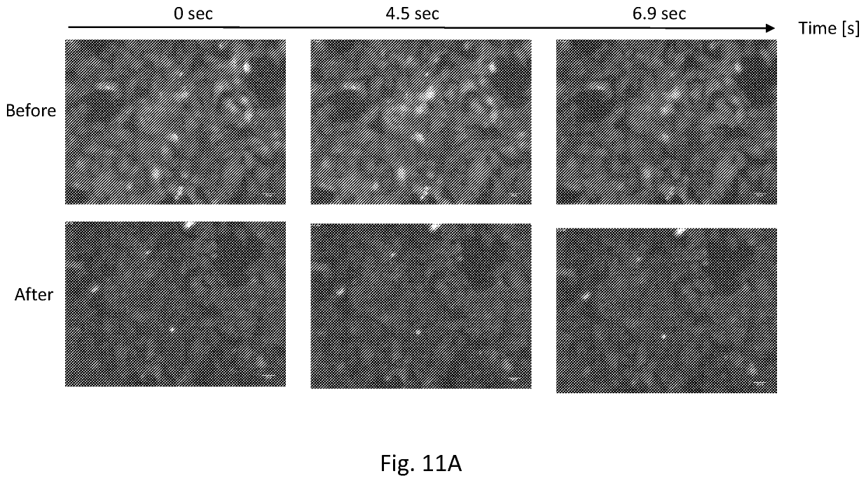

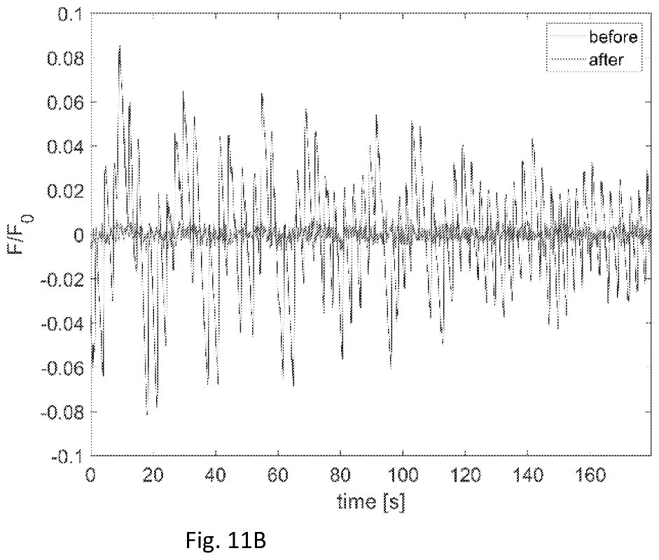

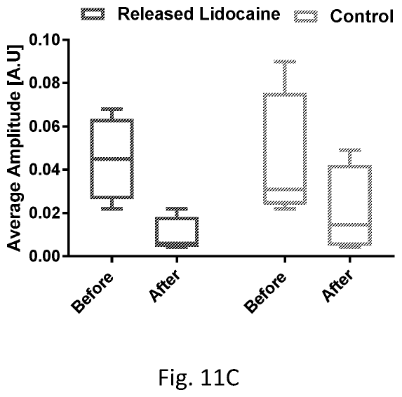

In accordance with various potential embodiments, and with reference to FIGS. 11A-11C, the released lidocaine was tested on cardiomyocytes. Lidocaine is known to have a cardiotoxic effect in high concentrations, and accordingly, HL-1 cardiomyocytes beating on a plate were imaged using calcium imaging before and after exposure to the released lidocaine (FIG. 11A). This cell line demonstrates synchronized calcium wave propagation when grown to sufficient cell density. When lidocaine released from the rubber bands was added to the cells, their rate of contraction dropped, and the fluorescence pattern changed dramatically as well, indicating a much lower level of calcium activity which is the basis for cardiomyocyte contraction (FIGS. 11B and 11C).

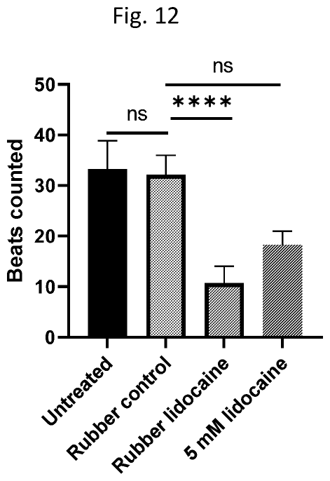

In accordance with various potential embodiments, and with reference to FIG. 12, rubber band elution was tested on a live animal model. As there are no readily available animal models, the effect of released lidocaine on the cardiac activity of zebrafish embryos was tested. Fish were grown in the presence of a drug-loaded rubber band, or a control rubber band that does not include lidocaine. When the drug-loaded rubber band was placed within the vicinity of zebrafish embryos, their heart rate decreased significantly. This effect was not observed in the unloaded rubber bands.

Other implementations include adornment of the surface of the device with micron sized barbs that increase the surface area of the device, to improve drug delivery, and also reduce the risk of slippage from the base of the hemorrhoid.

In other implementations, the anesthetic drug cargo may be an ester local anesthetic, for example, benzocaine, chloroprocaine, procaine, tetracaine, cocaine. In other implementations, the anesthetic drug cargo may be an amide local anesthetic, for example, lidocaine, mepivacaine, prilocaine, bupivacaine, ropivacaine. In other implementations, the anesthetic may be a selective enantiomer such as S(-) bupivacaine (levobupivacaine), or S(-) ropivacaine, both of which are reported to be mildly vasoconstrictive (a desirable property in the local application via rubber banding of hemorrhoids). In other implementations, the anesthetic may be n-butyl-p-amino-benzoate, which has been reported to have exceptionally long pharmacodynamic effect when formulated as a lipid suspension delivered epidurally. In other implementations, the anesthetic is a potent small molecule or peptide toxin such as saxitoxin, neosaxitoxin, tetrodotoxin, or other toxins such as the delta-atracotoxin peptide toxin, spider venom peptide phlotoxin 1, spider venom peptide Pn3a, ρ-conotoxin, δ-conotoxin, ω-conotoxin, a sea anemone peptide toxin, a scorpion toxins such as BmK AS, or other molecules capable of inhibiting voltage gated sodium channels necessary for painful sensation.

In other implementations, drug releasing elastic bands can also deliver vasoconstrictors, prothrombotic drug and sclerosing agents as the drug cargos of choice, for delivery of these (or other) drugs via elastic bands applied endoscopically for the control of gastrointestinal variceal bleeding. As discussed above, drug delivered locally via elution from the rubber band can achieve therapeutic effect at the intended site, with less concern about provoking untoward systemic effects.

In various potential embodiments, other possible uses for the device include more humane castration (elastration) and tail docking, two practices commonly used in animal husbandry using rubber bands to remove tails and testicles. Drug eluting rubber bands can reduce the pain and inflammation from various procedures and may thus reduce the risk of complications.

Non-limiting examples of various embodiments are disclosed herein. Features from one embodiments disclosed herein may be combined with features of another embodiment disclosed herein as someone of ordinary skill in the art would understand.

As utilized herein, the terms “approximately,” “about,” “substantially” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described without restricting the scope of these features to the precise numerical ranges provided. Accordingly, these terms should be interpreted as indicating that insubstantial or inconsequential modifications or alterations of the subject matter described and are considered to be within the scope of the disclosure.

For the purpose of this disclosure, the term “coupled” means the joining of two members directly or indirectly to one another. Such joining may be stationary or moveable in nature. Such joining may be achieved with the two members or the two members and any additional intermediate members being integrally formed as a single unitary body with one another or with the two members or the two members and any additional intermediate members being attached to one another. Such joining may be permanent in nature or may be removable or releasable in nature.

It should be noted that the orientation of various elements may differ according to other exemplary embodiments, and that such variations are intended to be encompassed by the present disclosure. It is recognized that features of the disclosed embodiments can be incorporated into other disclosed embodiments.

It is important to note that the constructions and arrangements of apparatuses or the components thereof as shown in the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter disclosed. For example, elements shown as integrally formed may be constructed of multiple parts or elements, the position of elements may be reversed or otherwise varied, and the nature or number of discrete elements or positions may be altered or varied. The order or sequence of any process or method steps may be varied or re-sequenced according to alternative embodiments. Other substitutions, modifications, changes and omissions may also be made in the design, operating conditions and arrangement of the various exemplary embodiments without departing from the scope of the present disclosure.

While various inventive embodiments have been described and illustrated herein, those of ordinary skill in the art will readily envision a variety of other mechanisms and/or structures for performing the function and/or obtaining the results and/or one or more of the advantages described herein, and each of such variations and/or modifications is deemed to be within the scope of the inventive embodiments described herein. More generally, those skilled in the art will readily appreciate that, unless otherwise noted, any parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, the technology described herein may be embodied as a method, of which at least one example has been provided. The acts performed as part of the method may be ordered in any suitable way unless otherwise specifically noted. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.” As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

1. A drug-eluting elastic medical apparatus comprising a composite of a therapeutic agent and an elastomer, the medical apparatus manufactured by:
dissolving the elastomer and the therapeutic agent in an organic solvent; and
evaporating the organic solvent while the elastomer reforms into the composite having a shape.

2. The medical apparatus of claim 1, wherein the organic solvent is evaporated while the elastomer reforms in a mold corresponding to the shape.

3. (canceled)

4. The medical apparatus of claim 1, wherein the shape corresponds to a rubber band comprising an outer diameter of 4 to 6 millimeters (mm), an inner diameter of 1 to 2 mm, a height of 1.5 to 2.5 mm.

5-7. (canceled)

8. The medical apparatus of claim 1, wherein the elastomer comprise polystyrene-block-polyisoprene-block-polystyrene, wherein the elastomer ranges from about 15 percentage by weight (wt %) polystyrene to 40 wt % polystyrene.

9-10. (canceled)

11. The medical apparatus of claim 1, wherein the elastomer consists substantially of a combination of at least two of (i) polyisoprene or derivatives thereof, (ii) polystyrene or derivatives thereof, (iii) polyisoprene or derivatives thereof, (iv) polyurethane or derivatives thereof, and (v) silicone or derivatives thereof.

12. The medical apparatus of claim 1, wherein the therapeutic agent comprises an analgesic, an anti-inflammatory agent, an anti-microbial agent, or a sclerotic agent.

13-15. (canceled)

16. The medical apparatus of claim 1, wherein the composite comprises a combination of two or more therapeutic agents.

17. (canceled)

18. The medical apparatus of claim 1, wherein the shape is a first shape, and wherein the medical apparatus is further manufactured by:
heating the composite to at least a melting temperature of the composite; and
dye-casting the composite into a second shape.

19. The medical apparatus of claim 1, wherein the medical apparatus comprises a rubber band manufactured to release the therapeutic agent through diffusion from pores in the rubber band.

20-24. (canceled)

25. The medical apparatus of claim 1, further comprising an elastic band applicator, wherein the elastic band applicator is a ligator.

26. (canceled)

27. A medical procedure comprising securing a drug-eluting elastic medical apparatus to a tissue of a subject to locally release a therapeutic agent into the tissue and/or its surroundings, the medical apparatus comprising a composite of the therapeutic agent and an elastomer, the medical apparatus manufactured by:
dissolving the elastomer and the therapeutic agent in an organic solvent; and
evaporating the organic solvent while the elastomer reforms into the composite having a shape.

28. The medical procedure of claim 27, wherein the medical procedure strangulates and necroses the tissue such that the tissue breaks off.

29. The medical procedure of claim 27, wherein the shape corresponds to a rubber band, wherein the medical procedure comprises banding of a hemorrhoid.

30-41. (canceled)

42. The medical procedure of claim 27, wherein the therapeutic agent comprises a combination of two or more therapeutic agents, the combination including at least two of an analgesic, an anti-inflammatory agent, an anti-microbial agent, or a sclerotic agent.

43-47. (canceled)

48. A method comprising manufacturing a drug-eluting elastic medical apparatus comprising a composite of a therapeutic agent and an elastomer by:
dissolving the elastomer and the therapeutic agent in an organic solvent; and
evaporating the organic solvent while the elastomer reforms into the composite having a shape.

49. The method of claim 48, wherein the organic solvent is evaporated while the elastomer reforms in a mold corresponding to the shape.

50-52. (canceled)

53. The method of claim 48, wherein the shape corresponds to a 3D printer filament, and wherein manufacturing the medical apparatus further comprises using the 3D printer filament to print the medical apparatus.

54-57. (canceled)

58. The method of claim 48, wherein the elastomer consists substantially of a combination of at least two of (i) polyisoprene or derivatives thereof, (ii) polystyrene or derivatives thereof, (iii) polyisoprene or derivatives thereof, (iv) polyurethane or derivatives thereof, and (v) silicone or derivatives thereof, wherein the elastomer comprises polystyrene-block-polyisoprene-block-polystyrene, wherein the elastomer ranges from about 15 percentage by weight (wt %) polystyrene to 40 wt % polystyrene.

59-62. (canceled)

63. The method of claim 48, wherein the shape is a first shape, and wherein manufacturing the medical apparatus further comprises:
heating the composite to at least a melting temperature of the composite; and
dye-casting the composite into a second shape.

64. The method of claim 48, wherein the medical apparatus comprises a rubber band that is manufactured to release the therapeutic agent through diffusion from pores in the rubber band, the method further comprising using the medical apparatus in a medical procedure that brings the medical apparatus into contact with a tissue of a subject to locally release the therapeutic agent into the tissue and/or its surroundings.

65-69. (canceled)
